(19) United States
(12) Patent Application Publication
(10) Pub. No.: US 2021/0393800 A1
(43) Pub. Date: Dec. 23, 2021

(54) PREPARATION AND APPLICATION OF SUPRAMOLECULAR SELF-ASSEMBLED HYALURONIC ACID HYDROGEL

(71) Applicant: PHI BioMed Inc., Gangnam-gu, Seoul (KR)

(72) Inventors: Sei Kwang HAHN, Haeundae-gu, Busan (KR); Mun Gu KIM, Pohang-si, Gyeongsangbuk-do (KR); Sang Hoon JEONG, Namdong-gu, Incheon (KR)

(21) Appl. No.: 17/287,398

(22) PCT Filed: Oct. 25, 2019
(86) PCT No.: PCT/KR2019/014211

§ 371 Date: Apr. 21, 2021

(30) Foreign Application Priority Data
Oct. 26, 2018 (KR) 10-2018-0129187
Oct. 24, 2019 (KR) 10-2019-0133082

(51) Int. Cl. A61K 47/69; A61K 47/61; A61K 47/54; A61K 38/18; A61K 9/06

(52) U.S. Cl. CPC A61K 47/6951; A61K 47/61; A61K 9/06; A61K 38/1808; A61K 47/54

(57) ABSTRACT

A hydrogel prepared through the supramolecular self-assembly of cyclodextrin and adamantane is described. The hydrogel can be filled with drugs and cells and used for various diseases. The hydrogel uses hyaluronic acid and thus can be applied to transdermal delivery, in vivo drug release, intractable disease treatment using stem cells, etc.

### TECHNICAL FIELD

The present invention relates to a supramolecular self-assembled hyaluronic acid hydrogel, a method of preparing the hydrogel, and applications of the hydrogel.

### BACKGROUND ART

Recently, various biomaterials have been developed for drug delivery, three-dimensional cell culture, and cell delivery. Among them, hydrogels have emerged as the most effective material that meets the above purposes, and various studies are being conducted thereon. The hydrogels are water-soluble polymers with a network structure and have properties similar to those of tissues because they are not dissolved in water and have a high moisture content. The hydrogels can be induced in the form of a gel by inducing a covalent bond between functional groups using a highly reactive chemical material. In recent years, attention has been focused on injectable hydrogels that can be filled with drugs or cells without denaturation and can be injected into the human body with minimal wounds. In order to develop the injectable hydrogels, studies using a method using chemical reactions resulting from an enzyme, a chemical material, and light, a method using external stimuli such as a temperature or pH change, and a method using physical changes resulting from a solvent change and swelling have been attempted.

As the representative method using chemical reactions, there is a method using a highly reactive chemical material, e.g., a method using the Michael addition which is a reaction between a thiol group and a double bond. There is PEGDA formed by polymerizing a polymer having a reactive group polymerizable with commercially available HyStem-C using a photoinitiator (i.e., formed using a photopolymerization method). HyStem has an advantage in that a hydrogel can be formed by introducing a thiol group into hyaluronic acid and simply mixing the same with PEGDA, and a product only consisting of a PEG derivative has an advantage in that a hydrogel can be formed within a short time of 5 minutes by adding a photoinitiator to PEGDA and performing UV irradiation. However, the photopolymerization requires the use of an initiator to induce the reaction, and most of the initiators commercially available to date have a problem of cytotoxicity. Also, since it is difficult for ultraviolet rays irradiated for the photopolymerization to pass through the skin, the practical use of the products as the injectable hydrogel is difficult. In addition, PEGDA, which is commonly used in both products, uses a double bond with good reactivity and thus has a disadvantage in that a double bond which does not participate in gel formation has a potential to cause toxicity when introduced into the body.

In addition, products called Corgel™ that uses an enzyme, which induces reactive oxygen species to cause a chemical reaction, such as horseradish peroxidase, and Gelite® that uses an ionic reaction in which an anionic polymer forms a chelate using electrostatic attraction with divalent or higher cations are commercially available. However, the method using an enzyme has a possibility of an immune rejection reaction by the enzyme because an external enzyme such as horseradish peroxidase is mixed immediately before injection and then injected, as compared with a method using an intrinsic in vivo enzyme. In addition, the method using an ionic reaction has a disadvantage in that it is difficult to maintain a gel state in vivo for a long period of time because the hardening of the gel depends on the concentration of cations, so this method is currently mainly used in research on development of a product for drug delivery.

As the method of forming a hydrogel using external stimuli, there is a method utilizing a temperature-sensitive system, and ReGel® that uses a triblock copolymer PLGA-PEG-PLGA, Pluronic® (Poloxamer) that uses a triblock copolymer PEO-PPO-PEO, and Matrigel™ that uses collagen derived from a sarcoma cell of an Engelbreth-Holm-Swarm (EHS) murine sarcoma are commercially available. In the temperature-sensitive system, a lower critical solution temperature (LCST) phenomenon occurs, and this phenomenon is specifically a phenomenon in which the product is present in a liquid state at low temperatures and becomes a solid state at a body temperature (37° C.). This has an advantage in that drug delivery and cell delivery can be easily attempted by storing the product in a liquid state at low temperatures and then injecting it into the living body to form a gel when necessary. Particularly, Matrigel™ can allow the survival period of cells to be extended due to having various types of cell growth factors. However, since such a temperature-sensitive injectable hydrogel is formed in a network structure with a physical bond forming micelles, it has a disadvantage in that the hydrogel cannot be stably maintained in vivo for a long period of time because of a large initial release amount and a high in vivo decomposition rate. In addition, in the case of Regel®, since a synthetic polymer is used, the decomposition product is partially cytotoxic, and since Matrigel™ is derived from murine cancer cells, there is a limitation in being practically used in humans.

As a pH-sensitive system, a product called Carbopol® that uses polyacrylic acid is commercially available. This product is present in a liquid state at pH 4 due to having low viscosity, and as pH increases, the ionized acrylic acid group gradually neutralizes and becomes hydrophobic, and thus physical bonding occurs due to hydrophobic reactions, resulting in an increase in viscosity, and the product is solidified at a physiologically active pH (pH 7.4). Therefore, since the product easily reaches a physiologically active pH (pH 7.4) even when injected in a liquid state, a gel can be simply formed. However, to inject the gel, the product needs to be used in an acidic environment of pH 4, so there is a disadvantage in that cells may be damaged during administration.

As described above, since the method of preparing an injectable hydrogel using chemical reactions may cause severe damage to cells carried in the hydrogel due to a highly reactive chemical material, several attempts have been made to form a hydrogel only using physical interactions without introduction of a functional group into a polymer, and products such as ReGel®, Matrigel™, Pluronic®, and the like are commercially available. However, since these products have a limitation in in vivo application as cell therapy products due to having the above-described disadvantages, there is a need to develop a safe injectable hydrogel which remains stable in vivo for an appropriate time, has excellent biocompatibility and biodegradability, exhibits less immune rejection reactions, and does not have toxicity.

### DISCLOSURE

### Technical Problem

The present invention is directed to providing a hydrogel prepared by supramolecular self-assembly of cyclodextrin and adamantane.

### Technical Solution

One aspect of the present invention provides a hydrogel prepared from: a hyaluronic acid-cyclodextrin derivative; and a hyaluronic acid-adamantane derivative.

Another aspect of the present invention provides a method of preparing a hydrogel, which includes mixing a hyaluronic acid-cyclodextrin derivative and a hyaluronic acid-adamantane derivative.

Still another aspect of the present invention provides a drug carrier including: a hyaluronic acid-cyclodextrin derivative; and a hyaluronic acid-adamantane derivative.

Yet another aspect of the present invention provides a method of decomposing a hydrogel, which includes adding cyclodextrin to the above-described hydrogel.

### Advantageous Effects

The present invention provides a hydrogel prepared by supramolecular self-assembly of cyclodextrin and adamantane.

The hydrogel according to the present invention can be used for various diseases by being filled with drugs and cells and can be applied to transdermal delivery, in vivo drug release, intractable disease treatment using stem cells, and the like by having hyaluronic acid.

### DESCRIPTION OF DRAWINGS

FIG. 1 shows a schematic diagram of the synthesis of a hyaluronic acid-cyclodextrin derivative.

FIG. 2 shows a nuclear magnetic resonance (NMR) analysis result of a hyaluronic acid-cyclodextrin derivative.

FIG. 3 shows a Fourier-transform infrared spectroscopy (FT-IR) analysis result of a hyaluronic acid-cyclodextrin derivative.

FIG. 4 shows a schematic diagram of the synthesis of a hyaluronic acid-adamantane derivative.

FIG. 5 shows an NMR analysis result of a hyaluronic acid-adamantane derivative.

FIG. 6 shows a FT-IR analysis result of a hyaluronic acid-adamantane derivative.

FIGS. 7 and 8 show rheological testing results of a hydrogel according to the present invention.

FIG. 9 shows an animal experiment result of a hydrogel according to the present invention.

FIGS. 10 and 11 are an image and graph illustrating the decomposition of a hydrogel according to the present invention, respectively.

FIG. 12 is a graph illustrating a protein release experiment result.

### MODES OF THE INVENTION

The present invention relates to a hydrogel prepared from: a hyaluronic acid-cyclodextrin derivative; and a hyaluronic acid-adamantane derivative.

Hereinafter, the hydrogel of the present invention will be described in more detail.

In the present invention, hyaluronic acid may be safely applied to the human body due to having not only biocompatibility and biodegradability but also transdermal delivery characteristics, and may be applied in a transdermal drug delivery system of various protein medicines including antigenic proteins and chemical medicines.

In the present invention, “hyaluronic acid (HA)” refers to a polymer having a repeat unit represented by the following General Formula 1 unless otherwise indicated, and is used with a meaning encompassing a salt or derivative of hyaluronic acid.

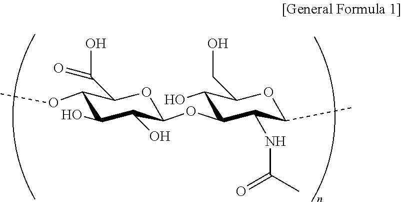

In General Formula 1, n may be an integer ranging from 50 to 10,000.

In the present invention, as the salt of hyaluronic acid, a tetrabutylammonium salt of hyaluronic acid (HA-TBA) may be used.

In the present invention, the “hyaluronic acid derivative” refers to all of the modified forms of hyaluronic acid based on the basic structure of hyaluronic acid of General Formula 1, into which a functional group such as an amine group, an aldehyde group, a vinyl group, a thiol group, an allyloxy group, N-succinimidyl-3-(2-pyridyldithio)propionate (SPDP), N-hydroxysuccinimide (NHS), or the like is introduced. For example, as the hyaluronic acid derivative, HA-diaminobutane, HA-hexamethylenediamine, HA-aldehyde, HA-adipic acid dihydrazide (HA-ADH), HA-2-aminoethyl methacrylate hydrochloride, HA-spermine, HA-spermidine, HA-SPDP, HA-NETS, or the like may be used.

Hyaluronic acid is present in most animals and is a linear polysaccharide polymer with biodegradability, biocompatibility, and no immune responses, and thus may be safely applied to the human body. Since hyaluronic acid plays a number of different roles in the body depending on its molecular weight, it may be used for a variety of uses.

As used herein, the hyaluronic acid, salt of hyaluronic acid, or derivative of hyaluronic acid is not limited in configuration thereof, but the molecular weight thereof preferably ranges from 10,000 to 3,000,000 dalton (Da).

In addition, in the present invention, “hydrogel” refers to a gel in which water is a dispersion medium unless otherwise indicated. The hydrogel may be formed by a hydrosol losing fluidity by cooling or by expanding a hydrophilic polymer with a three-dimensional network structure and a microcrystal structure by containing water. Most electrolyte polymer hydrogels exhibit high absorbability and are practically used as absorbent polymers in various fields. Some hydrogels undergo phase transition due to temperature, pH, or the like, and thus the expansion ratio thereof is discontinuously changed.

The hydrogel according to the present invention may be prepared from a hyaluronic acid-cyclodextrin derivative and a hyaluronic acid-adamantane derivative.

In the present invention, the hyaluronic acid-cyclodextrin derivative (HA-CD derivative) refers to a derivative in which hyaluronic acid and cyclodextrin are combined by an amide bond. Specifically, a carboxyl group of the hyaluronic acid and an amine group of the cyclodextrin may form an amide bond. In this case, one or more hydroxyl groups of the cyclodextrin are substituted with amine groups, and thus the amine group may form a bond.

The hyaluronic acid-cyclodextrin derivative may be represented by the following Chemical Formula 1.

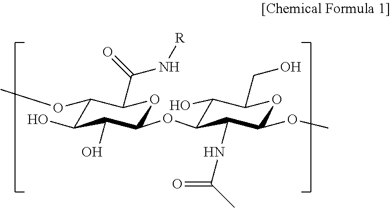

In Chemical Formula 1, R may be cyclodextrin.

In an embodiment, the hyaluronic acid-cyclodextrin derivative may be prepared by reacting hyaluronic acid and cyclodextrin.

Specifically, the hyaluronic acid-cyclodextrin derivative may be prepared by dissolving hyaluronic acid, a salt of hyaluronic acid, or a derivative of hyaluronic acid and cyclodextrin in a solvent and then reacting the same in the presence of a coupling reagent.

As the solvent, water, dimethyl sulfoxide (DMSO), or phosphate-buffered saline (PBS) may be used. As the coupling reagent, 4-(4,6-dimethoxy-1,3,5-triazin-2-yl)-4-methylmorpholinium chloride (DMTMM), N-(3-dimethylaminopropyl)-N′-ethylcarbodiimide hydrochloride (EDC), N-hydroxysuccinimide (NHS), pyridine, N′-tetramethyl-O-(1H-benzotriazol-1-yl)uronium hexafluorophosphate (HBTU), or benzotriazol-1-yloxy)tris(dimethylamino)phosphonium hexafluorophosphate (BOP) may be used. In addition, PBS or 2-(N-morpholino)ethanesulfonic acid (IVIES) may be used as a buffer during the reaction, and the reaction may be performed at 10 to 40° C., 20 to 30° C., or room temperature.

In the present invention, the hyaluronic acid-adamantane (CD-Ad) derivative refers to a derivative in which hyaluronic acid and adamantane are combined by an ester bond. Specifically, a hydroxyl group of the hyaluronic acid and a carboxyl group of the adamantane may form an ester bond. In this case, as the adamantane, adamantaneacetic acid (Ada-acetic acid) in which a carboxyl group is substituted may be used.

The hyaluronic acid-adamantane derivative may be represented by the following Chemical Formula 2.

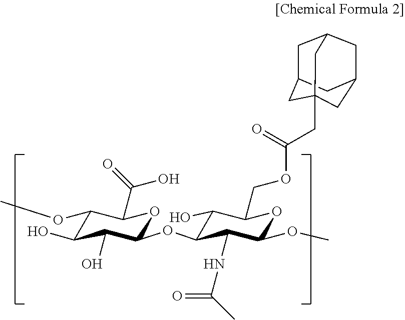

In an embodiment, the hyaluronic acid-adamantane derivative may be prepared by reacting hyaluronic acid and adamantane (Ada-acetic acid).

Specifically, the hyaluronic acid-adamantane derivative may be prepared by dissolving hyaluronic acid, a salt of hyaluronic acid, or a derivative of hyaluronic acid and adamantane in a solvent and then reacting the same in the presence of a reaction reagent.

As the solvent, water, dimethyl sulfoxide (DMSO), or phosphate-buffered saline (PBS) may be used. As the reaction reagent, a reagent that induces an ester reaction, such as 4-dimethylaminopyridine (4-DMAP), divinyl acetate (DVA), N,N′-dicyclohexylcarbodiimide (DCC), adamantane anhydride, or di-tert-butyl dicarbonate, may be used. In addition, PBS or 2-(N-morpholino)ethanesulfonic acid (MES) may be used as a buffer during the reaction. The reaction may be performed under vacuum.

In the present invention, the hydrogel may be prepared from the hyaluronic acid-cyclodextrin derivative and the hyaluronic acid-adamantane derivative, and specifically, the hydrogel may be prepared by a supramolecular reaction between cyclodextrin of the hyaluronic acid-cyclodextrin derivative and adamantane of the hyaluronic acid-adamantane derivative.

As used herein, the supramolecule refers to a molecular complex formed by assembling molecules or ions through a noncovalent bond such as a hydrogen bond, an electrostatic interaction, or a van der Waals interaction. Since representative noncovalent bonds that form a supramolecular structure are much weaker than covalent bonds, the structure of a supramolecular material may be easily changed depending on a surrounding environment. Therefore, this characteristic may be used to optionally adjust the shape of a material. As a representative principle for forming a supramolecular structure, there is self-assembly. Self-assembly refers to a phenomenon in which molecules are assembled through spontaneous interactions.

In the present invention, a supramolecular complex, that is, a hydrogel, may be prepared using cyclodextrin. The cyclodextrin is a cyclic oligosaccharide having a hydrophobic cavity formed by an α-1,4 bond of 6 to 8 glucose molecules and is classified into α-cyclodextrin having 6 glucose molecules, β-cyclodextrin having 7 glucose molecules, and γ-cyclodextrin having 8 glucose molecules. In this case, the molecular weight, hydrophobic cavity size, solubility, and the like of the cyclodextrin may vary depending on the number of glucose molecules forming the cyclodextrin.

The cyclodextrin has a structure in which hydroxyl groups bound to C2 and C3 are directed outward, and a hydroxyl group bound to C6 is also directed outward, as analyzed by X-rays, and thus the outer shell of the ring is entirely hydrophilic. On the other hand, a hydrogen ion and oxygen of an ether at C3 and C5 are positioned inside the structure of cyclodextrin, and thus the inner cavity is hydrophobic. Therefore, the hydrophilic outer shell of the entire structure is allowed to be dissolved well in a polar solvent such as water, while the hydrophobic pore that has the opposite nature to the outer shell is formed inside the structure. This enables the formation of a complex through a host-guest interaction which is the greatest characteristic of cyclodextrin.

A guest material forms a complex through structural fitting while going into the cyclodextrin pore with a certain size, and the height of the pore is identical, but the diameter and volume thereof vary according to the type of cyclodextrin. In the present invention, adamantane is used as the guest material. The adamantane has a structure in which 4 cyclohexane rings are condensed in a basket shape, is a highly symmetrical and stable compound, and may form a bond through a host-guest interaction with cyclodextrin.

That is, according to the present invention, the hydrogel may be prepared through a host-guest interaction between the cyclodextrin of the hyaluronic acid-cyclodextrin derivative and the adamantane of the hyaluronic acid-adamantane derivative.

In an embodiment, the content ratio of the hyaluronic acid-cyclodextrin derivative and the hyaluronic acid-adamantane derivative may be 1:0.1 to 1:10, 1:0.5 to 1:2, or 1:1.

In addition, in an embodiment, the hydrogel may be prepared by physically mixing the hyaluronic acid-cyclodextrin derivative and the hyaluronic acid-adamantane derivative.

In an embodiment, the hydrogel of the present invention may further include a useful substance selected from the group consisting of a drug, a fluorescent material, a radioisotope, a target-directing material, an imaging material, and a cell. The hydrogel including the useful substance may function as a drug carrier for delivering the useful substance.

The drug is a substance capable of inhibiting, suppressing, reducing, alleviating, delaying, preventing, or treating a disease or a symptom in animals including humans, and examples thereof include paclitaxel, doxorubicin, docetaxel, 5-fluoreuracil, oxaliplatin, cisplatin, carboplatin, berberine, epirubicin, doxycycline, gemcitabine, rapamycin, tamoxifen, Herceptin, Avastin, Tysabri, Erbitux, Campath, Zevalin, Humira, Mylotarg, Xolair, Bexxar, Raptiva, Remicade, siRNA, aptamers, interferons, insulin, Reopro, Rituxan, Zenapax, Simulect, Orthoclone, Synagis, erythropoietin, epidermal growth factors (EGFs), human growth hormones (hGHs), thioredoxin, Fel d1, Api m1, myelin basic protein, Hsp60, DnaJ, and the like.

The fluorescent material may be a fluorescent material typically used in the art to which the present invention belongs, and examples thereof include fluorescein, rhodamine, dansyl, Cy, anthracene, and the like.

The radioisotope may be ³H, ¹⁴C, ²²Na, ³⁵S, ³³P, ³²P, and ¹²⁵I.

The target-directing material is any material capable of selectively recognizing, binding to, or delivering a specific target material, and examples thereof include peptides such as arginine-leucine-aspartic acid (RGD), threonine-alanine-threonine (TAT), methionine-valine-Dmethionine (MVm), and the like, peptides that recognize a specific cell, antigens, antibodies, folic acid, nucleic acids, aptamers, and carbohydrates (e.g., glucose, fructose, mannose, galactose, ribose, and the like).

The imaging material is any material that can be detected through spectroscopy such as nuclear magnetic resonance (NMR), magnetic resonance imaging (MRI), positron emission tomography (PET), or computed tomography (CT) or through a microscope such as a fluorescence microscope, a confocal laser scanning microscope, or the like, and examples thereof include a Ga-complex, nanoparticles, carbon nanomaterials, and the like, but the present invention is not limited thereto. Examples of the Ga-complex include Ga-DTPA, Ga-DTPA-BMA, Ga-DOT, Ga-cyclam, and the like, examples of the nanoparticles include gold, silver, manganese, cadmium, selenium, tellurium, zinc, and the like, and preferably the nanoparticles are nanoparticles having a size of 1 to 200 nm, and examples of the carbon nanomaterials include single-walled nanotubes, multi-walled nanotubes, fullerenes, graphene, and the like.

In addition, the present invention relates to a drug carrier including a hyaluronic acid-cyclodextrin derivative and a hyaluronic acid-adamantane derivative.

Since the hydrogel according to the present invention is prepared by self-assembly of the above-described derivatives, the hydrogel may carry a useful substance, specifically, a drug, therein. Examples of the drug include the above-described types of drugs, that is, paclitaxel, doxorubicin, docetaxel, 5-fluoreuracil, oxaliplatin, cisplatin, carboplatin, berberine, epirubicin, doxycycline, gemcitabine, rapamycin, tamoxifen, Herceptin, Avastin, Tysabri, Erbitux, Campath, Zevalin, Humira, Mylotarg, Xolair, Bexxar, Raptiva, Remicade, siRNA, aptamers, interferons, insulin, Reopro, Rituxan, Zenapax, Simulect, Orthoclone, Synagis, erythropoietin, epidermal growth factors (EGFs), human growth hormones (hGHs), thioredoxin, Fel d1, Api m1, myelin basic protein, Hsp60, DnaJ, and the like.

In an embodiment, the useful substance may be easily carried in the hydrogel by mixing the hyaluronic acid-cyclodextrin derivative and the hyaluronic acid-adamantane derivative with the useful substance in the preparation of the hydrogel.

In an embodiment, in the preparation of the drug carrier, that is, in the carrying of the useful substance in the hydrogel, the useful substance may be used in the form of a HA-useful substance conjugate in which a useful substance is bound to hyaluronic acid or in the form of a HA-Ad-useful substance conjugate in which a useful substance is bound to a hyaluronic acid-adamantane derivative. Alternatively, the useful substance may be used by itself. The HA-useful substance conjugate may be formed by introducing an aldehyde group into hyaluronic acid and binding a useful substance (the useful substance may include an amine group or may be modified with an amine group) to the hyaluronic acid through an amine-aldehyde reaction. In addition, the HA-Ad-useful substance conjugate may be formed by introducing an aldehyde group into a hyaluronic acid-adamantane (HA-Ad) derivative and binding a useful substance (the useful substance may include an amine group or may be modified with an amine group) to the HA-Ad derivative through an amine-aldehyde reaction.

The drug carrier may be used for the purpose of delivering the useful substance in vivo or in vitro to animals including humans.

In addition, the present invention provides a pharmaceutical composition including the drug carrier according to the present invention. The pharmaceutical composition may further include a pharmaceutically acceptable carrier, a diluent, or the like and may be administered by any method known to those skilled in the art, for example, oral or parenteral administration, such as injection, infusion, transplantation. Examples of parenteral routes include intravascular, intratumoral, transmucosal, percutaneous, intramuscular, intranasal, intravenous, intradermal, subepidermal, intraperitoneal, intraventricular, intracranial, intravaginal, inhalational, rectal administration, and the like.

The pharmaceutical composition may be used by using the prepared hydrogel as it is or by formulating the hydrogel in a form suitable for the route of administration, that is, in a solid preparation, a liquid preparation, or a hydrogel.

In addition, the present invention relates to a method of decomposing a hydrogel, which includes adding cyclodextrin to the above-described hydrogel.

When cyclodextrin is added to the hydrogel of the present invention, the decomposition of the hydrogel occurs.

In this case, the cyclodextrin may be added in an amount of 2.5 to 5 parts by weight relative to the total weight (100 parts by weight) of the hydrogel.

Hereinafter, the present invention will be described in detail with reference to the following examples. However, the following examples are merely presented to exemplify the present invention, and the content of the present invention is not limited to the following examples.

### EXAMPLES

### Example 1. Preparation of Hydrogel

(1) Preparation of Hyaluronic Acid-Cyclodextrin (HA-CD) Derivative

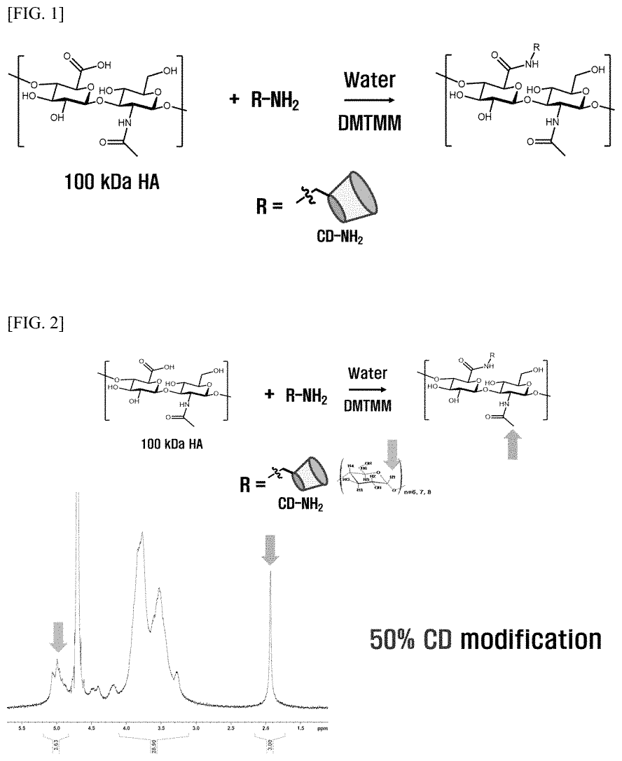

A schematic diagram of a process of synthesizing a hyaluronic acid-cyclodextrin derivative is shown in FIG. 1.

Specifically, 100 mg of hyaluronic acid and 282.5 mg of 4-(4,6-dimethoxy-1,3,5-triazin-2-yl)-4-methylmorpholinium chloride (DMTMM) were dissolved in 10 ml of a 2-(N-morpholino)ethanesulfonic acid (MES) buffer solution, mixed with 158.76 mg of cyclodextrin, and reacted at room temperature.

Afterward, unreacted DMTMM and unreacted cyclodextrin were removed through a dialysis process. The dialysis process was performed by adding 0.1 M sodium chloride on day 1 and adding water from day 2 to day 3. After the dialysis, lyophilization was performed.

(2) Preparation of Hyaluronic Acid-Adamantane (HA-Ad) Derivative

A schematic diagram of a process of synthesizing a hyaluronic acid-adamantane derivative is shown in FIG. 4.

First, a tetrabutylammonium hydroxide (TBA) salt of hyaluronic acid was prepared. Specifically, 12.5 g of DOWEX 50WX 8,400 was dispersed in 250 ml of water for 15 minutes to prepare a liquid dispersion. A process of precipitating the liquid dispersion and then removing a supernatant was repeated. Then, 24.5 ml of a TBA salt was added to the resulting liquid dispersion and then stirred. After the stirring, the resulting mixture was filtered through a filter, and only powder was extracted.

1 g of hyaluronic acid was dissolved in 100 ml of water, and the prepared powder was added thereto and stirred for 3 hours. The stirred mixture was filtered and then lyophilized to prepare a TBA salt of hyaluronic acid.

The TBA salt of hyaluronic acid, 123 mg of 1-adamantaneacetic acid, and 18.9 mg of 4-dimethylaminopyridine (4-DMAP) were mixed and then dissolved after adding 7.5 ml of dimethyl sulfoxide (DMSO) under vacuum. After the dissolution, 22.92 mg of di-tert-butyl dicarbonate was added.

Afterward, unreacted adamantaneacetic acid and unreacted DMAP were removed through a dialysis process. The dialysis process was performed by adding DMSO at a volume ratio of 20% to 5 L of water on day 1, adding 0.1 M sodium chloride on day 2, and adding water on day 3. After the dialysis, lyophilization was performed.

(3) Preparation of Hydrogel

The hyaluronic acid-cyclodextrin derivative prepared in the step (1) and the hyaluronic acid-adamantane derivative prepared in the step (2) were mixed in a ratio of 1:1 and uniformly mixed in a physical manner at room temperature to prepare a hydrogel.

### Experimental Example 1. Nuclear Magnetic Resonance (NMR) Analysis of Derivatives

(1) Method

The hyaluronic acid-cyclodextrin derivative prepared in the step (1) of Example 1 and the hyaluronic acid-adamantane derivative prepared in the step (2) of Example 1 were analyzed by NMR (DPX300, Bruker, Germany).

(2) Results

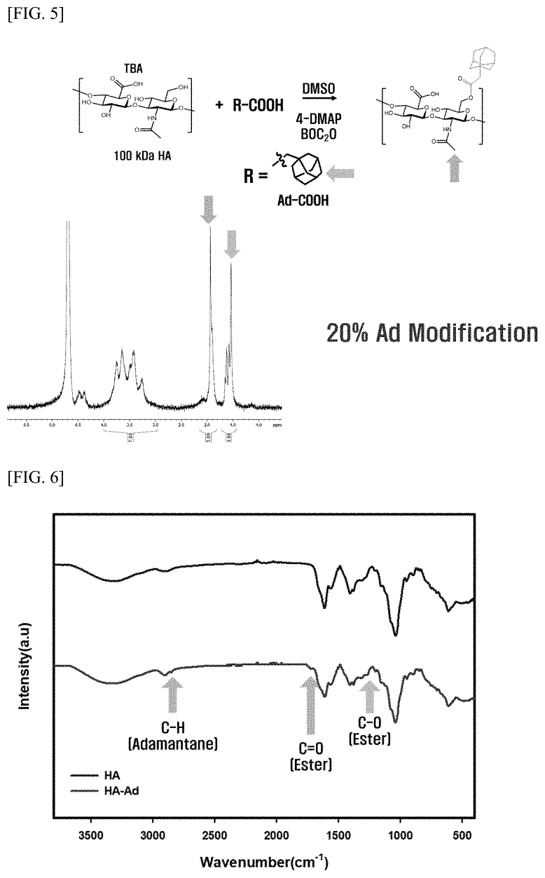

An NMR analysis result for the hyaluronic acid-cyclodextrin derivative is shown in FIG. 2, and an NMR analysis result for the hyaluronic acid-adamantane derivative is shown in FIG. 5.

First, as shown in FIG. 2, cyclodextrin peaks were detected. From this result, it can be seen that cyclodextrin is conjugated to hyaluronic acid.

In addition, as shown in FIG. 5, adamantane peaks were detected. From this result, it can be seen that adamantane is conjugated to hyaluronic acid.

### Experimental Example 2. Fourier-Transform Infrared Spectroscopy (FT-IR) Analysis of Derivatives

(1) Method

The hyaluronic acid-cyclodextrin derivative prepared in the step (1) of Example 1 and the hyaluronic acid-adamantane derivative prepared in the step (2) of Example 1 were analyzed by FT-IR (NICOLET 5700).

(2) Results

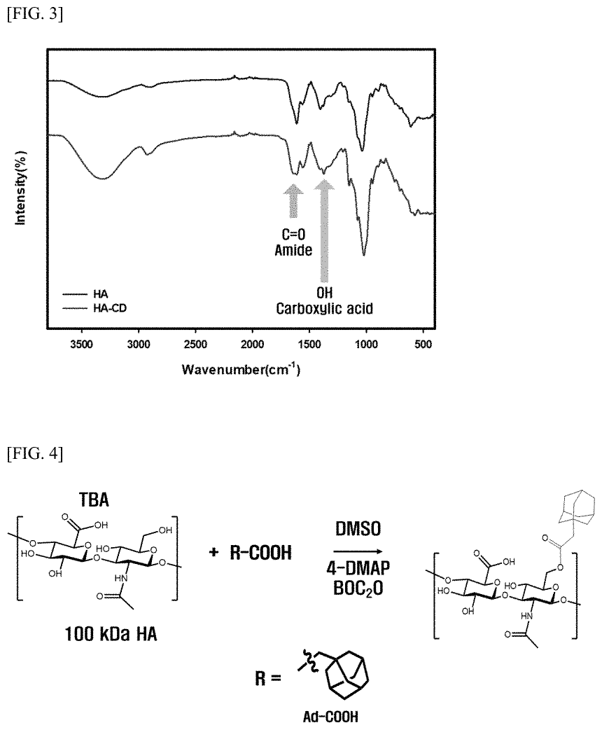

A FT-IR analysis result for the hyaluronic acid-cyclodextrin derivative is shown in FIG. 3, and a FT-IR analysis result for the hyaluronic acid-adamantane derivative is shown in FIG. 6.

First, as shown in FIG. 3, it can be seen from a result of analyzing a molecular structure by FT-IR that cyclodextrin is conjugated to hyaluronic acid by forming an amide bond.

In addition, as shown in FIG. 6, it can be seen that adamantane is conjugated to hyaluronic acid by forming an ester bond.

### Experimental Example 3. Measurement of Physical Properties of Hydrogel

(1) Method

Physical properties of the hydrogel prepared in the step (3) of Example 1 were measured.

Specifically, moduli and shear stress of the hydrogel were measured using a rheometer (MCR101, Anton Paar).

The storage modulus and loss modulus were measured while angular frequency was changed from 0.01 to 100 with strain fixed at 2%, and viscosity and shear stress were measured while a shear rate (1/s) was changed from 0.1 to 10000.

(2) Results

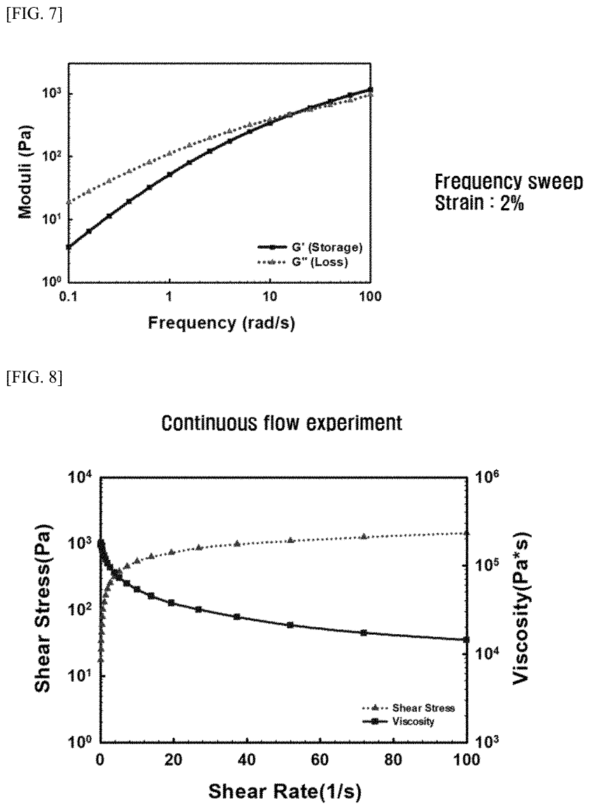

Measurement results are shown in FIGS. 7 and 8.

As shown in FIGS. 7 and 8, physical properties were changed according to shear stress and also changed according to a change in angular frequency with strain fixed at 2%. From these results, it can be seen that shear thinning occurs.

### Experimental Example 4. Animal Experiment

(1) Method

Each of the HA-CD and HA-Ad prepared in the steps (1) and (2) of Example 1 were dissolved at 5 wt % in PBS. 50 ul of the 5 wt % HA-CD and 50 ul of the 5 wt % HA-Ad were input into a syringe and uniformly mixed, and the mixture was subcutaneously injected into mice. The mice were sacrificed and dissected according to date, and then whether the hydrogel remained was checked.

(2) Results

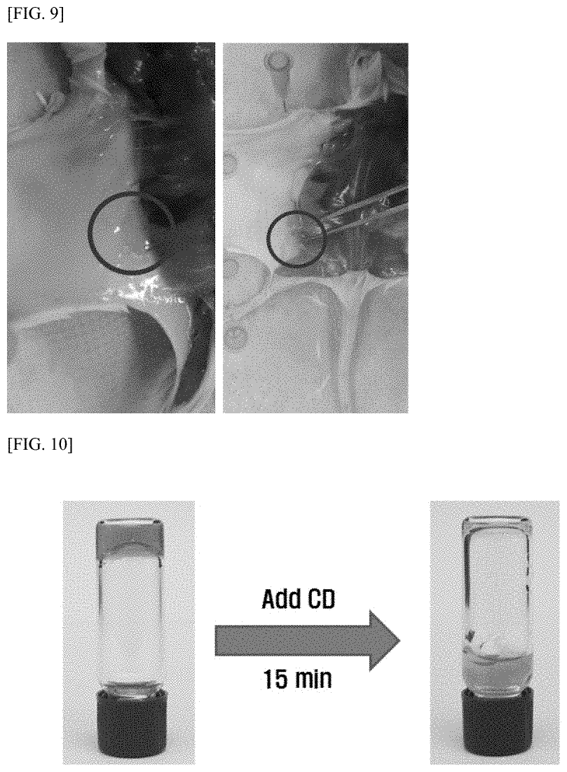

Results thereof are shown in FIG. 9.

As shown in FIG. 9, it can be seen that the hydrogel remains until day 28.

### Experimental Example 5. Cyclodextrin-Induced Hydrogel Decomposition Experiment

(1) Method

150 ul of the 5 wt % HA-CD and 150 ul of the 5 wt % HA-Ad were mixed to form a hydrogel, and 300 ul of a 50 mg/ml aqueous CD solution was then added onto the hydrogel. After 15 minutes, the vial containing the hydrogel was turned over, and whether the gel retained its form was checked to confirm the decomposition of the hydrogel.

(2) Results

Results thereof are shown in FIG. 10.

As shown in FIG. 10, it can be seen that, 15 minutes after the addition of cyclodextrin to the hydrogel, the hydrogel is decomposed and thus present in a liquid state (image on the right).

### Experimental Example 6. Enzyme-Induced Hydrogel Decomposition Experiment

(1) Method

150 ul of the 5 wt % HA-CD and 150 ul of the 5 wt % HA-Ad were mixed to form a hydrogel, and the hydrogel was input into an E-tube and centrifuged to remove air bubbles inside the gel.

1 ml of a solution made by dissolving 400 U/ml hyaluronidase in PBS was added thereto in an experimental group, and 1 ml of PBS was added thereto in a control group. Afterward, the concentration of decomposed hydrogel in a supernatant was checked according to date.

(2) Results

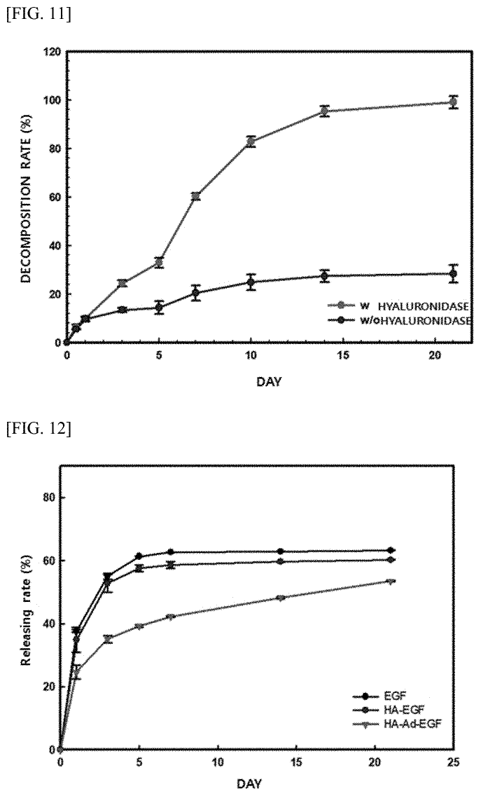

Results thereof are shown in FIG. 11.

As shown in FIG. 11, it can be seen that the degree of hyaluronidase-induced decomposition of the hydrogel in an experimental group (w hyaluronidase) is higher than that in a control group (w/o hyaluronidase).

### Experimental Example 7. Protein Release Experiment

(1) Method

Aldehyde groups were introduced to the hyaluronic acid-adamantane (HA-Ad) derivative and hyaluronic acid (HA) using sodium peroxidase.

The HA-Ad derivative and HA, to which the aldehyde group had been introduced, were allowed to react with an EGF protein using sodium cyanoborohydride to induce an aldehyde-amine reaction, thereby preparing conjugates. Through the aldehyde-amine reaction, a HA-Ad-EGF conjugate and a HA-EGF conjugate were prepared.

Each of EGF, the prepared HA-Ad-EGF conjugate, and the prepared HA-EGF conjugate was added at a concentration of 20 ug/ml to a 5 wt % hyaluronic acid-adamantane (HA-Ad) derivative solution. 50 ul of a 5 wt % hyaluronic acid-cyclodextrin (HA-CD) derivative solution was added to 50 ul of each of the resulting solutions and then mixed to prepare a hydrogel.

The hydrogel was input into an E-tube and centrifuged to remove air bubbles inside the gel. 500 ul of PBS was added to each hydrogel.

The concentration of EGF included in a supernatant was analyzed by high-performance liquid chromatography (HPLC) according to date.

(2) Results

Analysis results are shown in FIG. 12.

In FIG. 12, EGF represents a case where EGF was used in preparation of the hydrogel, HA-EGF represents a case where a HA-EGF conjugate was used, and HA-Ad-EGF represents a case where a HA-Ad-EGF conjugate was used.

As shown in FIG. 12, the amount of EGF released over time increased in the order of HA-Ad-EGF<HA-EGF<EGF. Since the same concentration of protein was added to each hydrogel, it can be seen that HA-Ad-EGF is able to release a constant concentration of protein most slowly for a long period of time.

A general drug carrier releases a large amount of drug at the early stage and an insignificant amount of drug at subsequent stages, whereas the drug carrier according to the present invention can steadily release the drug at a constant concentration.

### INDUSTRIAL APPLICABILITY

The hydrogel according to the present invention can be used for various diseases by being filled with drugs and cells and can be applied to transdermal delivery, in vivo drug release, intractable disease treatment using stem cells, and the like by having hyaluronic acid.

1. A hydrogel prepared from: a hyaluronic acid-cyclodextrin derivative; and a hyaluronic acid-adamantane derivative.

2. The hydrogel of claim 1, wherein the hyaluronic acid-cyclodextrin derivative is a derivative in which hyaluronic acid and cyclodextrin are combined by an amide bond.

3. The hydrogel of claim 1, wherein the hyaluronic acid-adamantane derivative is a derivative in which hyaluronic acid and adamantane are combined by an ester bond.

4. The hydrogel of claim 1, wherein a content ratio of the hyaluronic acid-cyclodextrin derivative and the hyaluronic acid-adamantane derivative is 1:0.1 to 1:10.

5. The hydrogel of claim 1, wherein the hydrogel is formed by a supramolecular reaction between the cyclodextrin of the hyaluronic acid-cyclodextrin derivative and the adamantane of the hyaluronic acid-adamantane derivative.

6. The hydrogel of claim 1, further comprising a useful substance selected from the group consisting of a drug, a fluorescent material, a radioisotope, a target-directing material, an imaging material, and a cell.

7. A method of preparing a hydrogel, comprising:
mixing a hyaluronic acid-cyclodextrin derivative and a hyaluronic acid-adamantane derivative.

8. A drug carrier comprising: a hyaluronic acid-cyclodextrin derivative; and a hyaluronic acid-adamantane derivative.

9. A method of decomposing a hydrogel, comprising:
adding cyclodextrin to a hydrogel prepared from a hyaluronic acid-cyclodextrin derivative and a hyaluronic acid-adamantane derivative.

10. The method of claim 9, wherein the cyclodextrin is added in an amount of 2.5 to 5 parts by weight relative to a total weight of the hydrogel.
